(19) United States
(12) Patent Application Publication
(10) Pub. No.: US 2025/0074971 A1
(43) Pub. Date: Mar. 6, 2025

(54) SINGLE-DOMAIN ANTIBODY AGAINST SVA AND USES THEREOF

(71) Applicant: Lanzhou Veterinary Research Institute Chinese Academy of Agricultural Sciences, Lanzhou (CN)

(72) Inventors: Shiqi SUN, Lanzhou (CN); Huichen GUO, Lanzhou (CN); Suyu MU, Lanzhou (CN); Shuzhen TAN, Lanzhou (CN); Hu DONG, Lanzhou (CN); Jianing WANG, Lanzhou (CN); Lingbo CHEN, Lanzhou (CN); Manyuan BAI, Lanzhou (CN); Yun ZHANG, Lanzhou (CN)

(21) Appl. No.: 18/824,847

(22) Filed: Sep. 4, 2024

(30) Foreign Application Priority Data
Sep. 5, 2023 (CN) 202311135472.3

(51) Int. Cl. C07K 16/10; G01N 33/569

(52) U.S. Cl. CPC C07K 16/1009; G01N 33/56983; C07K 2317/22; C07K 2317/24; C07K 2317/565; C07K 2317/569; C07K 2317/76; G01N 2333/01

(57) ABSTRACT

A single-domain antibody or an antigen-binding fragment thereof to Senecavirus A, the single-domain antibody or the antigen-binding fragment thereof being selected from 52 amino acid sequence groups, and each of the 52 amino acid sequence groups including a first variable domain of heavy chain VH-CDR-1, a second variable domain of heavy chain VH-CDR-2, and a third variable domain of heavy chain VH-CDR-3. The first variable domain of heavy chain VH-CDR-1 has an amino acid sequence represented by one of SEQ ID NO: 1-SEQ ID NO: 52. The second variable domain of heavy chain VH-CDR-2 has an amino acid sequence represented by one of SEQ ID NO: 53-SEQ ID NO: 104. The third variable domain of heavy chain VH-CDR-3 has an amino acid sequence represented by one of SEQ ID NO: 105-SEQ ID NO: 156.

### CROSS-REFERENCE TO RELATED APPLICATIONS

Pursuant to 35 U.S.C.§ 119 and the Paris Convention Treaty, this application claims foreign priority to Chinese Patent Application No. 202311135472.3 filed Sep. 5, 2023, the contents of which, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, MA 02142.

### INCORPORATION-BY-REFERENCE OF SEQUENCE LISTING

This application contains a sequence listing, which has been submitted electronically in XML file and is incorporated herein by reference in its entirety. The XML file, created on Aug. 6, 2024, is named LZSY-00801-UUS.xml, and is 140,296 bytes in size.

### BACKGROUND

The disclosure relates to the field of antibody engineering and pharmaceuticals, more particularly, to a single-domain antibody against SVA and its uses.

Senecavirus A (SVA) is a member of the Picornaviridae family and is known to cause vesicular symptoms in pigs, affecting their hooves and oral cavities. SVA is a non-enveloped, single-stranded positive-sense RNA virus with a genome of approximately 7.3 kilobases (kb). A 5′ untranslated region (5′ UTR) and a 3′ untranslated region (3′ UTR) are two segments of the viral RNA, respectively, located at the 5′ end and 3′ end of the genome. The 5′ UTR contains a secondary RNA structure called an internal ribosome entry site (IRES). The IRES allows the virus to initiate translation in a cap-independent manner (Hales et al., 2008). The genome contains an open reading frame (ORF) that encodes a polyprotein. The polyprotein contains a Leader protein (L), a P1 region consisting of four polypeptides VP1, VP2, VP3, and VP4, a P2 region consisting of four polypeptides 2A, 2B, and 2C, and a P3 region consisting of four polypeptides 3A, 3B, 3C and 3D. The polyprotein is cleaved by 3C protease, a viral-encoded protease, into the 12 viral proteins according to the standard picornavirus layout of L-4-3-4. The four polypeptides (VP4, VP2, VP3, and VP1) of the P1 region are structural proteins that form the viral capsid, involved in receptor binding and cell entry. The Leader protein (L) and the seven polypeptides (2A, 2B, 2C, 3A, 3B, 3C, and 3D) are the non-structural proteins that are essential for viral replication and are involved in virus-host interactions and immune evasion, affecting virulence and pathogenesis. SVA closely resembles Foot-and-Mouth Disease (FMD), causing acute and severe illness in livestock, particularly pigs. The clinical similarities between SVA and FMD can lead to confusion in diagnosis and management, resulting in significant financial losses for the livestock industry. In addition to pigs, cattle can also act as natural hosts for SVA. Interestingly, asymptomatic SVA-positive cases have been reported, further complicating control measures. The best strategy for controlling SVA includes developing vaccines and diagnostic reagents. However, progress has been hindered by the lack of precise identification of SVA-neutralizing epitopes and the absence of high-affinity antibodies. Therefore, there is a need for the development of high-affinity, specific, and sensitive antibodies.

Antibodies are immunoglobulins produced by plasma cells derived from B lymphocytes in response to antigen stimulation. Antibodies are Y-shaped molecules composed of four polypeptide chains: two heavy chains (H chains) and two light chains (L chains). The L chains are bound to the H chains by disulfide bonds and non-covalent interactions. Structurally, the top of the Y shape contains two variable regions (V), each capable of binding tightly to a specific part of an antigen called an epitope. They consist of the shape's base, and the lower arms consist of constant domains (C). V gene and C gene encode the variable region (V) and the antibody's constant region (C). During lymphocyte differentiation, the V gene and C genes are rearranged to produce diverse heavy chains and light chains, which are then transcribed and translated.

In 1993, Hamers-Casterman discovered that camel serum contains many molecules similar to immunoglobulin G (IgG) in addition to conventional antibodies.

The molecules naturally lack the light chains and the part of the constant region of the heavy chains found in conventional antibodies but still retain the ability to bind to antigens. The molecules are known as heavy-chain antibodies (HCAbs). In other words, camels produce two types of antibodies: conventional antibodies and HCAbs. The conventional antibodies are dimers composed of two identical heavy chains and two identical light chains. HCAbs (VHHs or single-domain antibodies) are dimers composed solely of the variable domain of the heavy chain of conventional antibodies without any light chains. Compared to the antigen-binding domains of mouse and human antibodies (−150 kDa), camel VHHs (single-domain antibodies) retain full antigen specificity at around 15 kDa. Conventional antibodies have a large molecular weight, are unstable in vitro, and tend to aggregate. In contrast, single-domain antibodies (VHHs) have a small molecular weight, strong stability, and good solubility, easy expression, and low production costs. Therefore, single-domain antibodies (VHHs) have garnered significant attention for overcoming most of the shortcomings of conventional antibodies. Specific single-domain antibodies can be screened by immunizing animals of the camelid family and using phage display. Due to their high specificity, small molecular size, good tissue penetration, and ease of modification, single-domain antibodies (VHHs) are applied in diagnostics and disease treatment. Most production of single-domain antibodies uses prokaryotic expression systems, which are cost-effective and high-yield. However, the prokaryotic expression systems may result in misfolding and loss of biological activity. Hence, developing an efficient method for producing single-domain antibodies is of great significance.

Single-domain antibodies (VHHs) have been applied to various viruses, including HIV, influenza, rabies, poliovirus, FMD virus, rotavirus, hepatitis C virus, SARS-CoV-1, MERS-CoV, and SARS-CoV-2. While several conventional murine monoclonal antibodies targeting SVA have been studied, most of the conventional murine monoclonal antibodies, despite their specificity, lack neutralizing activity.

### SUMMARY

To solve the aforesaid problems, the first objective of the disclosure is to provide a single-domain antibody or an antigen-binding fragment thereof specifically binding to Senecavirus A (SVA). The single-domain antibody or antigen-binding fragment thereof contains a heavy chain encompassing a variable region. The variable region contains three complementarity-determining regions (CDRs): CDR-1 (VH-CDR-1), CDR-2 (VH-CDR-2), and CDR-3 (VH-CDR-3). VH-CDR-1 has an amino acid sequence represented by one of SEQ ID NO: 1 to SEQ ID NO: 52. VH-CDR-2 has an amino acid sequence represented by one of SEQ ID NO: 53 to SEQ ID NO: 104. VH-CDR-3 has an amino acid sequence represented by one of SEQ ID NO: 105 to SEQ ID NO: 156. In certain embodiments, the single-domain antibody or antigen-binding fragment thereof comprises: (1) CDR-1 comprising the amino acid sequence represented by SEQ ID NO: 1, CDR-2 comprising the amino acid sequence represented by SEQ ID NO: 53, and CDR-3 comprising the amino acid sequence represented by SEQ ID NO: 105.

In a class of this embodiment, preferably, VH-CDR-1 has an amino acid sequence represented by one of SEQ ID NO: 2, SEQ ID NO: 4, SEQ ID NO: 5, SEQ ID NO: 8, SEQ ID NO: 10, SEQ ID NO: 15, SEQ ID NO: 27, SEQ ID NO: 30, SEQ ID NO: 34, SEQ ID NO: 42, SEQ ID NO: 47, and SEQ ID NO: 52; VH-CDR-2 has an amino acid sequence represented by one of SEQ ID NO: 54, SEQ ID NO: 56, SEQ ID NO: 57, SEQ ID NO: 60, SEQ ID NO: 62, SEQ ID NO: 67, SEQ ID NO: 79, SEQ ID NO: 82, SEQ ID NO: 86, SEQ ID NO: 94, SEQ ID NO: 99, and SEQ ID NO: 104; and VH-CDR-3 has an amino acid sequence represented by one of SEQ ID NO: 106, SEQ ID NO: 108, SEQ ID NO: 109, SEQ ID NO: 112, SEQ ID NO: 114, SEQ ID NO: 119, SEQ ID NO: 131, SEQ ID NO: 134, SEQ ID NO: 138, SEQ ID NO: 146, SEQ ID NO: 151, and SEQ ID NO: 156.

In a class of this embodiment, preferably, VH-CDR-1 has an amino acid sequence represented by one of SEQ ID NO: 2, SEQ ID NO: 5, SEQ ID NO: 10, SEQ ID NO: 27, and SEQ ID NO: 47; VH-CDR-2 has an amino acid sequence represented by one of SEQ ID NO: 54, SEQ ID NO: 57, SEQ ID NO: 62, SEQ ID NO: 79, and SEQ ID NO: 99; and VH-CDR-3 has an amino acid sequence represented by one of SEQ ID NO: 106, SEQ ID NO: 109, SEQ ID NO: 114, SEQ ID NO: 131, and SEQ ID NO: 151.

In a class of this embodiment, the heavy chain of the antigen-binding fragment further contains a constant region selected from porcine IgG, human IgG, and/or chicken IgY.

In a class of this embodiment, the constant region is isolated from IgG1, IgG2a, IgG2b, and/or IgG4.

In a class of this embodiment, the antigen-binding fragment contains a functional fragment binding to an antigen.

In a class of this embodiment, VH-CDR-1 has an amino acid sequence represented by one of SEQ ID NO: 1 to SEQ ID NO: 52, an amino acid sequence having at least 80%, 85%, 90%, 91%, 92%, 93%, 94%, 95%, 96%, 97%, 98%, or 99% sequence identity to one of SEQ ID NO: 1 to SEQ ID NO: 52, or a sequence variant having no more than 8 amino acid substitutions from one of SEQ ID NO: 1 to SEQ ID NO: 52; VH-CDR-2 has an amino acid sequence represented by one of SEQ ID NO: 53 to SEQ ID NO: 104, an amino acid sequence having at least 80%, 85%, 90%, 91%, 92%, 93%, 94%, 95%, 96%, 97%, 98%, or 99% sequence identity to SEQ ID NO: 53 to SEQ ID NO: 104, or a sequence variant having no more than 8 amino acid substitutions from one of SEQ ID NO: 53 to SEQ ID NO: 104; and VH-CDR-3 has an amino acid sequence represented by one of SEQ ID NO: 105 to SEQ ID NO: 156, an amino acid sequence having at least 80%, 85%, 90%, 91%, 92%, 93%, 94%, 95%, 96%, 97%, 98%, or 99% sequence identity to SEQ ID NO: 105 to SEQ ID NO: 156, or a sequence variant having no more than 8 amino acid substitutions from one of SEQ ID NO: 105 to SEQ ID NO: 156.

In a class of this embodiment, the single-domain antibody is derived from a camelid single-domain antibody.

The second objective of the disclosure is to provide a nucleic acid molecule encoding the single-domain antibody or antigen-binding fragment thereof.

The third objective of the disclosure is to provide a vector or host cell comprising the nucleic acid molecule.

The fourth objective of the disclosure is to provide a chimeric antibody comprising the single-domain antibody or antigen-binding fragment thereof.

The fifth objective of the disclosure is to provide a composition comprising the single-domain antibody or antigen-binding fragment thereof, the nucleic acid molecule, the vector or host cell, and/or the chimeric antibody.

The sixth objective of the disclosure is to provide a method for diagnosis, prevention, and/or treatment of viral infections comprising administering a patient in need thereof the single-domain antibody or antigen-binding fragment thereof, the nucleic acid molecule, the vector or host cell, the chimeric antibody, and/or the composition.

In a class of this embodiment, the method is used for treating and/or inhibiting diseases caused by SVA infection.

The seventh objective of the disclosure is to provide a kit for detecting SVA.

The kit comprises an instruction manual and a reagent for detecting the single-domain antibody or antigen-binding fragment thereof, the nucleic acid molecule, the vector or host cell, the chimeric antigen receptor (CAR), a nucleic acid fragment encoding the CAR, a vector containing the nucleic acid fragment, a transformed immune cell expressing the CAR, and/or the composition.

The following advantages are associated with the disclosure.

The single-domain antibody or antigen-binding fragments thereof has a low immunogenicity risk and can effectively block SVA virus infection in cells, exhibiting significant SVA neutralizing activity. Therefore, the antibody sequences and the expression vectors of the disclosure have excellent applications in identifying SVA neutralizing epitopes, prevention or treatment, and diagnosis.

### BRIEF DESCRIPTION OF THE DRAWINGS

FIG. 1A is workflow of immunization of camels with SVA antigen using freund's adjuvant;

FIG. 1B is workflow of phage-display screening for single-domain antibodies specifically binding to SVA antigen;

FIG. 2 is a vector map of pNFCG1-EB containing Fc-VHH according to one example of the disclosure;

FIG. 3 shows the binding affinity and specificity of single-domain antibodies against SVA according to one example of the disclosure; and

FIG. 4 shows the neutralizing activity of single-domain antibodies against SVA.

### DETAILED DESCRIPTION

To further illustrate the disclosure, embodiments detailing the single-domain antibody against SVA or uses thereof are described below. It should be noted that the following embodiments are intended to describe and not to limit the disclosure.

The terms “antibody” and “immunoglobulin”, as described herein, are used interchangeably to refer to the same molecular structures. In conventional antibodies, two heavy chains are bound to each other by disulfide bonds, and each heavy chain is also bound to a light chain by disulfide bonds. There are two types of light chains: lambda (k) chains and kappa (x) chains. The heavy chain defines the class or isotypes of the antibodies. There are five isotypes: IgM, IgD, IgG, IgA, and IgE. The isotypes determine the functional activity of the antibodies. Each light chain and heavy chain consists of different sequences of regions. Light chains are composed of two regions: a variable region (VL) and a constant region (CL). Heavy chains are composed of four regions: a variable region (VH) and three constant regions (CH1, CH2, and CH3, collectively referred to as CH). The variable regions (VL and VH) of both the heavy chain (VH) and the light chain (VL) determine the antigen-binding recognition and specificity. The constant regions of both the light chain (CL) and the heavy chain (CH) confer crucial biological functions, including antibody assembly, secretion, placental transfer, complement fixation, and Fc receptor binding. Fv region is the N-terminal portion of the Fab region of the immunoglobulin, consisting of the variable regions of both the light chains and the heavy chains. The specificity of the antibodies depends on the structural complementarity between the antigen-binding site and the antigenic determinant. The antigen-binding site is mainly composed of residues from hypervariable regions, also known as CDRs.

The term “CDR”, as described herein, refers to the amino acid sequences that collectively determine the binding affinity and specificity of the natural binding site in the natural Fv region of the antibody or the immunoglobulin. Conventional immunoglobulins have three CDRs in each of the heavy chains and the light chains, labeled H-CDR1, H-CDR2, H-CDR3 for the heavy chains, and L-CDR1, L-CDR2, L-CDR3 for the light chains. In the disclosure, the single-domain antibody contains only three CDRs in the variable region of the heavy chain, and 52 sets of CDR1, CDR2, and CDR3 are screened.

The term “nucleic acid molecule” or “nucleic acid fragment”, as described herein, refers to any segment or segments of nucleic acids present in a polynucleotide, such as DNA or RNA fragments.

The term “antigen-binding fragment”, as described herein, refers to a molecule that contains an antigen-binding region capable of binding to a target antigen, such as proteins or polypeptides.

The term “variant”, as described herein, refers to antibodies that have been modified by 1-20 amino acid substitutions, deletions, and/or insertions in the target antibody region (such as the variable region of the heavy chain or the light chain, or the CDR regions of the heavy chain or the light chain), while substantially retaining the biological properties of the unmodified antibody. In one aspect, the disclosure provides variants of the antibodies described herein. In one example, the antibody variant retains at least 60%, 70%, 80%, 90%, or 100% of the biological activity (such as antigen-binding capacity or neutralizing capacity) of the unmodified antibody. The modifications can be applied individually or in combination to the variable region of the heavy chain, the variable region of the light chain, or each CDR region.

The term “chimeric antibody”, as described herein, refers to a recombinant protein that incorporates variable regions, including CDRs, derived from an antibody from one species, while the constant regions are sourced from a human antibody. Alternatively, the constant regions of the chimeric antibody may be obtained from other species, such as cats or dogs.

The term “sequence identity”, as used herein, refers to the degree of similarity between two or more biological sequences. The similarity is assessed within a sliding window that compares individual nucleotides in nucleic acids or amino acids in proteins. The “percent sequence identity” can be calculated as follows: aligning the two sequences within the sliding window to achieve the optimal alignment; identifying positions where nucleotide bases (e.g., A, T, C, G, I) or amino acid residues (e.g., Ala, Pro, Ser, Thr, Gly, Val, Leu, Ile, Phe, Tyr, Trp, Lys, Arg, His, Asp, Glu, Asn, Gln, Cys, and Met) are identical in both sequences; counting a total number of matching positions; dividing the total number of matching positions by a total number of positions in the sliding window (i.e., the size of the sliding window); and multiplying the result by 100 to obtain the percent sequence identity. The optimal alignment for determining the percent sequence identity can be achieved in various methods and software tools, such as BLAST, BLAST-2, ALIGN, or Megalign (DNASTAR) software. Those skilled in related art can adjust parameters and select suitable algorithms to achieve the optimal alignment over the full-length sequence being compared or within specific sequence regions of interest.

The term “vector”, as used herein, refers to a construct capable of introducing one or more genes or sequences of interest into a host cell and, preferably, to facilitate the expression of the genes or sequences within the host cell. The vectors include, but are not limited to, viral vectors, naked DNA or RNA expression vectors, plasmids, cosmids, or bacteriophage vectors, DNA or RNA expression vectors combined with cationic condensing agents, DNA or RNA expression vectors encapsulated in liposomes, and certain eukaryotic cells, such as production cells.

The terms “host cell”, as used herein, are used interchangeably to refer to cells that have been introduced with exogenous nucleic acids, including progeny of the cells. Host cells include “transformants” and “transformed cells,” which encompass primary transformed cells and progeny derived therefrom, regardless of the number of passages. Progeny may not be identical in nucleic acid content to the parent cell and may contain mutations. Cells that exhibit the same function or biological activity as those screened or selected from initially transformed cells are included.

The terms “neutralizing antibodies”, as used herein, are used interchangeably to refer to antibodies that bind to or interact with a target antigen and prevent the target antigen from binding to a binding partner, such as a receptor, thereby inhibiting or blocking the biological response that would result from the interaction of the target antigen with the binding partner.

The experimental methods and materials mentioned in the following examples are conventional and can be obtained through conventional commercial sources unless specified otherwise.

### Example 1 Screening of VHH Antibodies Against SVA

### 1. Preparation of SVA and Camel Immunization

1.1 Two healthy camels of the right age, designated S-1 #and S-2 #, were selected for the immunization process.

1.2 5 mL of peripheral blood was collected from each camel to isolate serum; the serum was used as a negative control for antibody titer test.

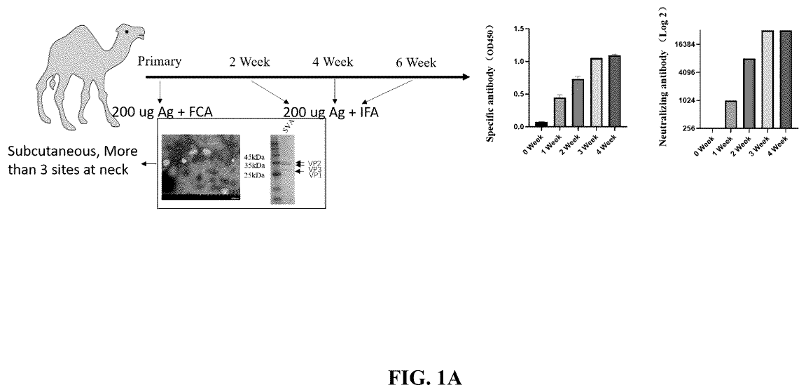

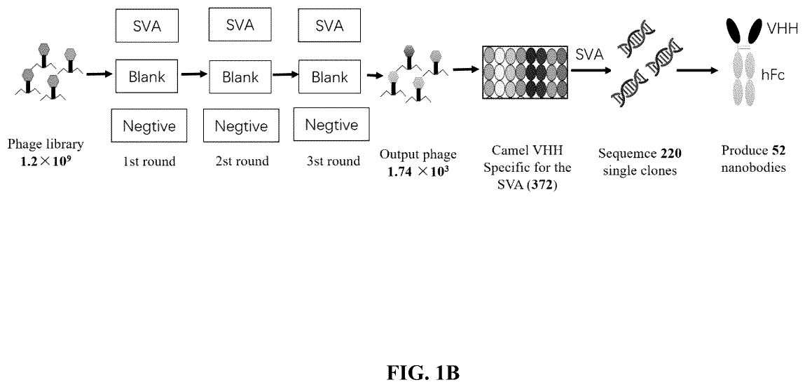

1.3 As depicted in FIG. 1, 400 g of SVA antigen was mixed with an equal volume of Freund's adjuvant and thoroughly emulsified; the resulting emulsion was administered subcutaneously at multiple sites along the neck of each camel; the initial immunization employed Freund's complete adjuvant; for subsequent booster immunizations, Freund's incomplete adjuvant was used; and the immunizations were administered at 14-day intervals.

Specifically, as depicted in FIG. 1A, the SVA antigen was purified and emulsified with Freund's complete adjuvant and Freund's incomplete adjuvant; and the resulting emulsion was administered subcutaneously at more than three sites along the neck at 0, 2, 4, and 6 weeks.

1.4 As depicted in FIG. 1, two weeks after the fourth immunization, 5 mL of whole blood was collected from each camel to isolate serum; the serum was then analyzed to determine the antibody titers.

1.5 200 ml of peripheral blood was collected from each camel; peripheral blood mononuclear cells (PBMCs) were isolated from the peripheral blood using the Ficoll density gradient centrifugation method and used to construct a camelid VHH antibody library; the two camel VHH antibody libraries from the two camels were combined to form a comprehensive library.

### 2. Construction of a Camel Phage-Displayed VHH Antibody Library

2.1 PBMCs were lysed with TriZol reagent to extract total RNA. Oligo-dT primers were used for reverse transcription PCR to prepare complementary DNA (cDNA).

2.2 In the first round of PCR, the cDNA was used as the template along with two primers: an upstream primer (SEQ ID NO: 157:

GTC.CTG.GCT.GCT.CTT.CTA.CAA.GG) that binds to the variable region of the heavy chain of the VHH antibody; and a downstream primer (SEQ ID NO: 158: GGT.ACG.TGC.TGT.TGA.ACT.GTT.CC) that binds to the constant region (CH2) of heavy chain of the VHH antibody. The PCR reaction yielded a 600 bp DNA band; the DNA band was used as a template in the second round of PCR; and the second round of PCR used primers specific for VHH antibody genes.

2.3 Restriction sites were added to two VHH-specific primers used in the second round of PCR to ensure that the VHH gene can be easily inserted into the vector: the second round of PCR employed the two VHH-specific primers:

a forward primer with a restriction site:
(SEQ ID NO: 159)
AACATGCCATGACTCGCGGCTCAACCGG CCATGGCTGA K GT B CAG
CTGCAG GC GTCTGG R GGAGG;
and
a reverse primer with a restriction site:
(SEQ ID NO: 160)
GTTATTATTATTCAGATTATTAGT
GCGGCCGCTGGAGACGGTGACC W GGGTCC.

The second round of PCR reaction yielded a VHH gene; a vector used for phage display was selected and digested with the same restriction enzymes that correspond to the restriction sites on the two specific primers; the VHH gene were also digested with the same restriction enzymes and ligated into the pre-digested vector using T4 DNA ligase; the ligation product was precipitated with ethanol to remove salts; and the purified ligation product was introduced into TG1 phage component cells via electroporation to construct a phage-displayed VHH antibody library with a capacity of 1.2×10⁹ different clones, each containing a different VHH gene.

### 3. Screening and Sequencing of VHH Antibodies Using Phage ELISA

3.1 Coating: A target protein was diluted in a coating buffer with a pH of 9.6; 50 μL of the diluted target protein solution was added to each well of the ELISA plate; and the ELISA plate was then placed in a humidified chamber and incubated at 37° C. for 1 hour, or alternatively, at 4° C. overnight.

3.2 Blocking: the unbound diluted coating buffer was discarded from the wells; the ELISA plate was inverted onto a clean paper towel and gently tapped to remove any residual liquid; the ELISA plate was washed three times with PBS; 300 μL of a blocking buffer was added into each well of the ELISA plate; and the ELISA plate was incubated at 37° C. for 1 hour.

3.3 Primary antibody incubation: the unbound phage solution was discarded from the wells; the ELISA plate was inverted onto a clean paper towel and gently tapped to remove any residual liquid; and the ELISA plate was washed three times with PBS; HRP-conjugated anti-M13 phage antibody was diluted 1:1000 in 1% Milk-PBS and added (50 L/well); and the ELISA plate was then incubated at 37° C. for 1 hour.

3.4 Color development: after incubation, the unbound HRP-conjugated anti-M13 phage antibody was discarded; the ELISA plate was inverted onto a clean paper towel and gently tapped to remove any residual liquid; the ELISA plate was washed four times with PBS; TMB (3,3′,5,5′-tetramethylbenzidine) substrate solution was added (50 μL/well); 2M H₂SO₄ was added to each well to stop the reaction; and absorbance of the stopped reaction was measured at 450 nm using a microplate reader.

3.5 The page clones that exhibited a color change were selected and sequenced.

Results: After immunizing the two camels with the target antigen (SVA) for three to four weeks, the VHH antibodies against SVA were detected in the blood of the two camels. The antibody titer was measured as 1:2,560,000, indicating a high concentration of the VHH antibodies. The phage-displayed VHH antibody library was constructed from RNA extracted from peripheral blood lymphocytes of the two camels. The phase-displayed VHH antibody library had a capacity of 1.2×10⁹ CFU/ml (as shown in FIGS. 1A and 1i). The phage-displayed VHH antibody library was screened for VHH antibodies against SVA particles (the target antigen). For control purposes, FMDV (Foot-and-Mouth Disease Virus) particles were used as a negative control, and wells without any antigen were used as a blank control. Three rounds of screening were conducted, and a total of 1.74×10³ phages were output. From the output phages, 372 individual phage clones were selected and tested using phage ELISA. The phage ELISA test identified 220 positive clones that specifically bound to the SVA particles. The 220 positive clones were analyzed using Sanger sequencing, and 52 unique VHH antibody sequences were identified (as shown in Tables 1-3). The 52 VHH antibody sequences were fused with human Fe region, and the fusion proteins were expressed in 293T cells (as depicted in FIG. 1B).

TABLE 1
Amino acid sequences of CDR-1 in VHH antibodies.

| CDR Sequence | Sequence Name | Amino acid sequence |
|---|---|---|
| CDR-1 | SEQ ID NO: 1 | YIKDINRLG |
| | SEQ ID NO: 2 | YTDSRFCLA |
| | SEQ ID NO: 3 | VTTCSNNMS |
| | SEQ ID NO: 4 | YAYIRYIGYSSSYCMG |
| | SEQ ID NO: 5 | YTFSSRPCMG |
| | SEQ ID NO: 6 | NTYSNTEYMG |
| | SEQ ID NO: 7 | YTYSSNCMG |
| | SEQ ID NO: 8 | VIYSSACLA |
| | SEQ ID NO: 9 | STDCSTYDMR |
| | SEQ ID NO: 10 | YTYCNYDMN |
| | SEQ ID NO: 11 | YCIG |
| | SEQ ID NO: 12 | PTYSTDCIG |
| | SEQ ID NO: 13 | YTYRSYCMG |
| | SEQ ID NO: 14 | YPYSSNCMG |
| | SEQ ID NO: 15 | LIFSDCTMA |
| | SEQ ID NO: 16 | YTYSSNCMG |
| | SEQ ID NO: 17 | VATSRLSMA |
| | SEQ ID NO: 18 | YTYSSNCMG |
| | SEQ ID NO: 19 | YIASLNCMA |
| | SEQ ID NO: 20 | YTFSSNCVG |
| | SEQ ID NO: 21 | FTARSYCMG |
| | SEQ ID NO: 22 | YQFSNGHVA |
| | SEQ ID NO: 23 | YTYSSGCMG |
| | SEQ ID NO: 24 | HTYCGNDRS |
| | SEQ ID NO: 25 | YTYKRRCMG |
| | SEQ ID NO: 26 | YPSSNHCMG |
| | SEQ ID NO: 27 | YIERHYCMG |
| | SEQ ID NO: 28 | YTVSSDCMG |
| | SEQ ID NO: 29 | FTSSINGMA |
| | SEQ ID NO: 30 | DTYSTVCLG |
| | SEQ ID NO: 31 | FRFIYKDYCMG |
| | SEQ ID NO: 32 | YTDSSYCMG |
| | SEQ ID NO: 33 | FTFSTYHMG |
| | SEQ ID NO: 34 | ATYATDCMG |
| | SEQ ID NO: 35 | YMSGNYCVG |
| | SEQ ID NO: 36 | FSYNNKCVA |
| | SEQ ID NO: 37 | YISSVSCMG |
| | SEQ ID NO: 38 | YAASANCMA |
| | SEQ ID NO: 39 | YDFSCMG |
| | SEQ ID NO: 40 | SIFSSCRMG |
| | SEQ ID NO: 41 | FPYSTVCMG |
| | SEQ ID NO: 42 | YIYSRSCMG |
| | SEQ ID NO: 43 | YMDNTYCIG |
| | SEQ ID NO: 44 | YTYSTHCMG |
| | SEQ ID NO: 45 | YSFSGYCIG |
| | SEQ ID NO: 46 | YIYSASCMG |
| | SEQ ID NO: 47 | YISNDYCMG |
| | SEQ ID NO: 48 | YPSSNHCMG |
| | SEQ ID NO: 49 | YTLSPYCMG |
| | SEQ ID NO: 50 | YTYCNYDMS |
| | SEQ ID NO: 51 | FTFSHYDMR |
| | SEQ ID NO: 52 | DTYSTVCLG |

TABLE 2
Amino acid sequences of CDR-2 in VHH antibodies

| CDR Sequence | Sequence Name | Amino acid sequence |
|---|---|---|
| CDR-2 | SEQ ID NO: 53 | TIRTVVGGSTYYTDSVKG |
| | SEQ ID NO: 54 | SIDSSGMTTYADSVKG |
| | SEQ ID NO: 55 | SIGVGTARYADSVKG |
| | SEQ ID NO: 56 | TIEPNGSASYSDSVKG |
| | SEQ ID NO: 57 | LIGRDGSTWYANSVKG |
| | SEQ ID NO: 58 | TIRMVVGGSTYYADSVKG |
| | SEQ ID NO: 59 | AIYTAGGNTYYADSVKG |
| | SEQ ID NO: 60 | RIDGYGNTRYEDSVKG |
| | SEQ ID NO: 61 | QSRTDGTTTYANAVKG |
| | SEQ ID NO: 62 | GLVYGGSTTYADSVKG |
| | SEQ ID NO: 63 | SIHVATGITYSADSVKG |
| | SEQ ID NO: 64 | QIASDGARRYADSVKG |
| | SEQ ID NO: 65 | TVFIGGDSTYYADSVKG |
| | SEQ ID NO: 66 | SIDSGGGTTYSASALE |
| | SEQ ID NO: 67 | SINADGGTPTYTDSVKG |
| | SEQ ID NO: 68 | AIYTGTGSTYYVDSVKG |
| | SEQ ID NO: 69 | AVNAGDGGTYYADSVKG |
| | SEQ ID NO: 70 | TIHTEYDSPYYASAVKG |
| | SEQ ID NO: 71 | YIGSGDGSTDYADSVKG |
| | SEQ ID NO: 72 | RYDGSGDPRYADSVKG |
| | SEQ ID NO: 73 | GIGFDGSTNYADSAKG |
| | SEQ ID NO: 74 | AIYAGGPVYTDSVKG |
| | SEQ ID NO: 75 | TSDSDGRTSYADSVKG |
| | SEQ ID NO: 76 | SIDSVGNTWYADSVKG |
| | SEQ ID NO: 77 | SIYTGGSSTYYADSVKG |
| | SEQ ID NO: 78 | SIDSVGITTYADSVLG |
| | SEQ ID NO: 79 | TVAYEGSTTYAESVKG |
| | SEQ ID NO: 80 | SIDNIGQTNYGDSVKG |
| | SEQ ID NO: 81 | AISSDGGVTDYADSVKG |
| | SEQ ID NO: 82 | VIDVDGKIVYADSIEG |
| | SEQ ID NO: 83 | VIDSDGRTAYANYVAG |
| | SEQ ID NO: 84 | AIGSDGGTSYADSVKG |
| | SEQ ID NO: 85 | AIDKYGTTYYRDSVKG |
| | SEQ ID NO: 86 | AYSRGTALYDDSVRG |
| | SEQ ID NO: 87 | VIEGGGSTRYAAAVKD |
| | SEQ ID NO: 88 | FIYTGDSSTDYFDSVKG |
| | SEQ ID NO: 89 | VISAADGSTDYDDSVKG |
| | SEQ ID NO: 90 | SIYVGGRTYYGDSVKG |
| | SEQ ID NO: 91 | SIRSFGGVYYADTVKG |
| | SEQ ID NO: 92 | SVSDDGITTYAEAVKG |
| | SEQ ID NO: 93 | TIYTAFGSTYYADSVKG |
| | SEQ ID NO: 94 | TIYTGSGSPWYADSVKG |
| | SEQ ID NO: 95 | AIGSVGSTSYADSVKG |
| | SEQ ID NO: 96 | YYYTGSGSTYYTDSVKG |
| | SEQ ID NO: 97 | RFDRDGRTTYAESVKG |
| | SEQ ID NO: 98 | VISPGDGSTDYADSVKG |
| | SEQ ID NO: 99 | ALHTGFGSTYYPDSVKG |
| | SEQ ID NO: 100 | SIDSVGTTTYADSVLG |
| | SEQ ID NO: 101 | VIDSTGSTKYAHSVKG |
| | SEQ ID NO: 102 | GIESFGDTSYADSVKG |
| | SEQ ID NO: 103 | GINSDGGDTYYADSVKG |
| | SEQ ID NO: 104 | VIDVDGKIVYADSIEG |

TABLE 3
Amino acid sequences of CDR-3 in VHH antibodies

| CDR | | |
|---|---|---|
| Sequence | Sequence Name | Amino acid sequence |
| CDR-3 | SEQ ID NO: 105 | GLGLYPSLSAAEYNY |
| | SEQ ID NO: 106 | RSRAWYEECSWVNKYSW |
| | SEQ ID NO: 107 | FCPNGGLWSGY |
| | SEQ ID NO: 108 | RAAINDCYGAPGNFDY |
| | SEQ ID NO: 109 | DPEYSASWCRDRRYRA |
| | SEQ ID NO: 110 | AMGLYPSLSRNEYNY |
| | SEQ ID NO: 111 | EIDPIWSTFTCGDWEADFSY |
| | SEQ ID NO: 112 | STTGWSCPVSLRMFEY |
| | SEQ ID NO: 113 | VGFGCRTGTVRNFNY |
| | SEQ ID NO: 114 | TTRGGWCPSGYDY |
| | SEQ ID NO: 115 | PTTYYCPSWEYESMFRW |
| | SEQ ID NO: 116 | RRGHPVWGCRAPDFDDYNF |
| | SEQ ID NO: 117 | QTGYTGSWCTEVASYGY |
| | SEQ ID NO: 118 | GERACFWNCFRTGWRNAD |
| | SEQ ID NO: 119 | YYCRFLGFSSF |
| | SEQ ID NO: 120 | RVPRSSSSWCSSLRRDEYVY |
| | SEQ ID NO: 121 | YFLGGDNLSRSQYNY |
| | SEQ ID NO: 122 | KAWGGDWCSRLDNYNY |
| | SEQ ID NO: 123 | DRISSTWCRFREYVS |
| | SEQ ID NO: 124 | GWALGWPATCDYSY |
| | SEQ ID NO: 125 | DFFRGSWCTGERAPTLRN |
| | SEQ ID NO: 126 | GLFFGAIWYDELRYFY |
| | SEQ ID NO: 127 | GLFFGAIWYDELRYFY |
| | SEQ ID NO: 128 | VCTGGSGPTKPV |
| | SEQ ID NO: 129 | KSDYWTWCNDGYSD |
| | SEQ ID NO: 130 | GSRAWYENCGRTVRRYSD |
| | SEQ ID NO: 131 | RTTYFCTPRANDFTY |
| | SEQ ID NO: 132 | QQFCTVGSRLSLDRDWYNY |
| | SEQ ID NO: 133 | DWYGSWYEVRRTKAYYY |
| | SEQ ID NO: 134 | GRKRHWALPTCGGMRPDY |
| | SEQ ID NO: 135 | TPVHPVFGQCRLVAGAFDY |
| | SEQ ID NO: 136 | NPGYLYSVVGYCYPAAPRNTH |
| | SEQ ID NO: 137 | SPGYGGPWYDSTWYNL |
| | SEQ ID NO: 138 | GNRAWYERSCQYND |
| | SEQ ID NO: 139 | RSRAWYESSCGNVGRFTW |
| | SEQ ID NO: 140 | SLAPSGSWCRARYYGY |
| | SEQ ID NO: 141 | DRRCYSGSWCLWRREYNY |
| | SEQ ID NO: 142 | SVSGGCWNFHPPFYEWRY |
| | SEQ ID NO: 143 | DNSYYCDVDKYDHKY |
| | SEQ ID NO: 144 | DHYHLIFRHNLCGR |
| | SEQ ID NO: 145 | ALLGTCCTDLLDGYNY |
| | SEQ ID NO: 146 | EPSFCTRSRRESDFRY |
| | SEQ ID NO: 147 | RFRSWYESCYTNQKYIY |
| | SEQ ID NO: 148 | LRAGYGGSWYECAISRANEYVH |
| | SEQ ID NO: 149 | VRPLCYRYCPPLGGAASDYNQ |
| | SEQ ID NO: 150 | DRRGSSGSWCLWRRAYSW |
| | SEQ ID NO: 151 | ARYSGSWCIVRGAYNHGY |
| | SEQ ID NO: 152 | GPRAWYENCGRTVRRYNA |
| | SEQ ID NO: 153 | APGRPTDLNTCMIAIEAPGAMRSAEI |
| | SEQ ID NO: 154 | NTRGGWCPSGFDY |
| | SEQ ID NO: 155 | GTIGISEP |
| | SEQ ID NO: 156 | GRKRHWALPTCGGMRPDY |

### 4. Small-Scale Expression of VHH Antibodies

4.1 Construction of expression vectors: The VHH gene was inserted into the pNFCG1-EB vector to construct a recombinant plasmid.

4.2 Cell preparation: Human embryonic kidney cells (293 cells) were collected, resuspended in culture medium to achieve a concentration of 1×10⁶ cells/mL, and incubated at 37° C. with 5% CO₂ to form a cell suspension.

4.3 Preparation of transfection complex: 1 μg of the recombinant plasmid was diluted in 40 μL of KPM medium and mixed; 5 μL of T1 transfection regent (in a 1:5 ratio with the recombinant plasmid) was diluted in 40 μL of KPM medium, mixed, and incubated at room temperature for 5 minutes; after incubation, the diluted T1 transfection regent was added to the diluted recombinant plasmid solution, mixed, and incubated at room temperature for 30 minutes to form a transfection complex.

4.4 Transfection: The transfection complex was added to the cell suspension, mixed, and incubated at 37° C. with 5% CO₂ to form transfected cells.

4.5 Supernatant collection: After 72 hours of incubation, the supernatant was collected and analyzed to detect and measure the activity and expression levels of the VHH antibody produced by the transfected cells.

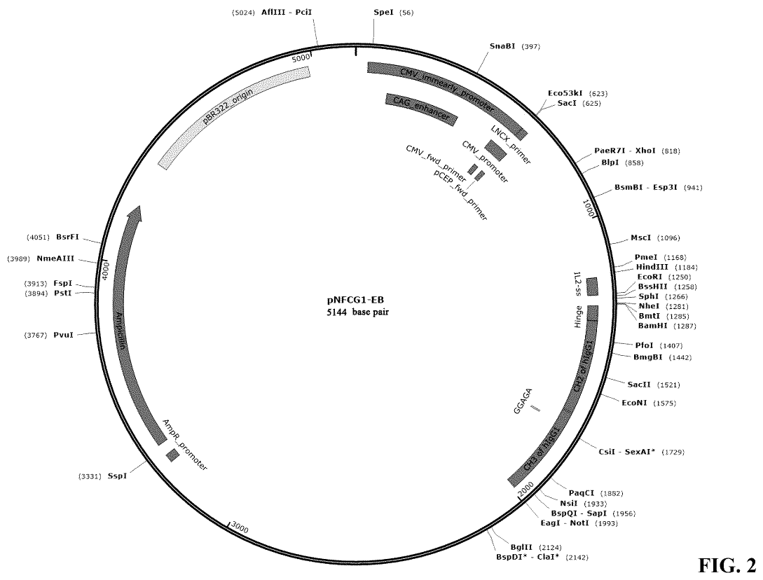

The 52 unique VHH sequence were respectively inserted into the pTT5 eukaryotic vector using specific restriction sites, NcoI and NotI. The pTT5 eukaryotic vector included a secretion signal peptide IL2 and a human IgG1-Fc tag. The 293F cells were adjusted to a concentration of 1×10⁷ cells/mL. The recombinant plasmid (pTT5 vector with VHH sequences) was transfected into 293F cells. After 3 days of incubation, the supernatant was collected by centrifugation at 4000×g for 30 minutes. The VHH antibodies, fused with the human IgG1-Fc tag, were purified using Protein A columns. The purification was carried out according to the manufacturer's instructions provided by GE Healthcare (as illustrated in FIG. 2). The binding activity of the VHH antibodies to the SVA antigen was analyzed using ELISA technology.

### Example 2 Validation of VHH Antibodies Against SVA

### 1. Detection of Specificity and Binding Activity of VHH Antibodies

1.1 Coating: Antigen S was diluted to a concentration of 5 μg/mL in a carbonate buffer with a pH of 9.6; 100 μL of the diluted antigen S was added to each well of a 96-well ELISA plate; and the 96-well ELISA plate was then incubated overnight at 4° C.

1.2 Blocking: The unbound coating buffer was discarded from the wells; the 96-well ELISA plate was washed three times with PBST; each well was blocked with 300 μL of a 4% skim milk in PBS; and the 96-well ELISA plate was incubated at 37° C. for 2 hours.

1.3 Sample addition: The unbound blocking solution was discarded; the 96-well ELISA plate was washed three times with PBST; samples, along with PBS and medium for the control group, were added to the wells (100 μL/well); and the 96-well ELISA plate was then incubated at 37° C. for 1 hour.

1.4 Secondary antibody incubation: After incubation, the unbound samples was discarded, and the 96-well ELISA plate was washed three times with PBST; the goat anti-human IgG-HRP (Fc) antibody, diluted 1:5000, was added (100 μL/well); and the 96-well ELISA plate was then incubated at 37° C. for 1 hour.

1.5 Color development: The unbound goat anti-human IgG-RP (Fc) antibody was discarded; the 96-well ELISA plate was washed five times with PBST; TMB substrate solution was added (100 μl/well) and developed in the dark.

1.6 Termination: After color development, 50 μl of 2M hydrochloric acid (HCl) was added to each well to stop the reaction.

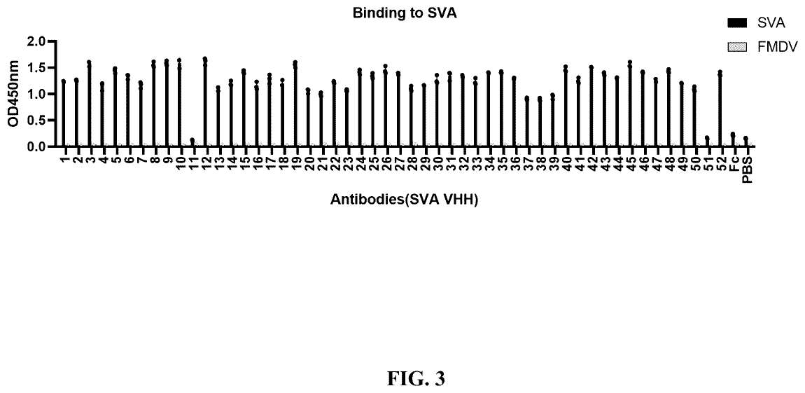

1.7 Reading: The absorbance of the stopped reaction was measured at 450 nm (A450) using a microplate reader (the results were shown in FIG. 3 and Table 4).

To measure the binding specificity of different VHH antibodies to SVA, an ELISA was performed. Specifically, the ELISA plate was first coated with SVA at a concentration of 1 μg/mL; additionally, FMDV was coated onto the same ELISA plate at the same concentration (1 pg/mL) to serve as a negative control; and each VHH antibody was then added to the wells at a concentration of 1 μg/ml.

Results: As shown in FIG. 3, expression in 293F cells revealed that out of 52 tested VHH antibodies, only VHH-11 and VHH-51 did not bind to SVA. The remaining 50 VHH antibodies exhibited binding activity to SVA and did not cross react with FMDV, indicating good specificity.

TABLE 4
Binding specificity and activity
of VHH antibodies to SVA and FMDV

| Antibody ID | SVA | FMDV |
|---|---|---|
| 1 | 1.236 | 0.050 |
| 2 | 1.259 | 0.051 |
| 3 | 1.577 | 0.050 |
| 4 | 1.151 | 0.050 |
| 5 | 1.443 | 0.050 |
| 6 | 1.329 | 0.052 |
| 7 | 1.169 | 0.050 |
| 8 | 1.557 | 0.053 |
| 9 | 1.586 | 0.049 |
| 10 | 1.589 | 0.049 |
| 11 | 0.120 | 0.058 |
| 12 | 1.616 | 0.050 |
| 13 | 1.102 | 0.052 |
| 14 | 1.201 | 0.052 |
| 15 | 1.417 | 0.054 |
| 16 | 1.154 | 0.055 |
| 17 | 1.286 | 0.050 |
| 18 | 1.196 | 0.051 |
| 19 | 1.553 | 0.050 |
| 20 | 1.056 | 0.049 |
| 21 | 0.993 | 0.054 |
| 22 | 1.224 | 0.057 |
| 23 | 1.065 | 0.055 |
| 24 | 1.415 | 0.050 |
| 25 | 1.344 | 0.049 |
| 26 | 1.452 | 0.052 |
| 27 | 1.387 | 0.050 |
| 28 | 1.104 | 0.056 |
| 29 | 1.163 | 0.050 |
| 30 | 1.270 | 0.052 |
| 31 | 1.347 | 0.050 |
| 32 | 1.349 | 0.050 |
| 33 | 1.234 | 0.049 |
| 34 | 1.401 | 0.051 |
| 35 | 1.409 | 0.052 |
| 36 | 1.290 | 0.050 |
| 37 | 0.905 | 0.052 |
| 38 | 0.893 | 0.057 |
| 39 | 0.948 | 0.063 |
| 40 | 1.466 | 0.054 |
| 41 | 1.254 | 0.049 |
| 42 | 1.508 | 0.048 |
| 43 | 1.387 | 0.049 |
| 44 | 1.305 | 0.048 |
| 45 | 1.554 | 0.050 |
| 46 | 1.413 | 0.050 |
| 47 | 1.248 | 0.050 |
| 48 | 1.434 | 0.050 |
| 49 | 1.206 | 0.050 |
| 50 | 1.094 | 0.051 |
| 51 | 0.169 | 0.053 |
| 52 | 1.380 | 0.050 |
| Fc | 0.218 | 0.054 |
| PBS | 0.154 | 0.053 |

### 2. Detection of Neutralizing Activity of VHH Antibodies

A neutralization assay was performed as follows: the VHH antibodies were prepared at a concentration of 1 μg/mL, with serial dilutions starting from 1:2; each dilution was repeated across four sample wells; the diluted VHH antibodies were incubated with 100 Tissue Culture Infectious Dose (TCID50) of the CH-HuB-2017 virus at 37° C. for 1 hour; 50 μL of IBRS-2 cells (at a concentration of 10⁵ cells/mL) was added to each well of a 96-well plate; the 96-well plate was then incubated at 37° C. with 5% CO₂ for 72 hours; the wells were examined using an inverted microscope to observe cytopathic effects; and the neutralizing antibody titers were calculated using the Reed-Muench method.

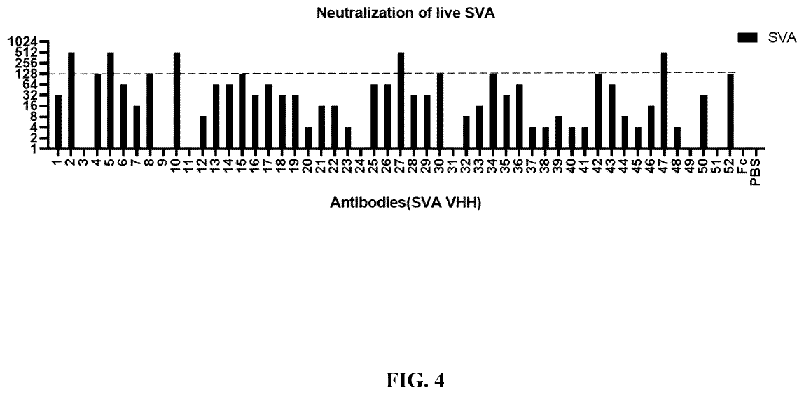

Results: The VHH antibodies with an initial concentration of 1 μg/mL were tested for their ability to neutralize SVA. The VHH antibodies with high neutralizing activity were identified following 2-fold serial dilutions and indicated by dashed lines (as shown in FIG. 4).

As shown in Table 5, out of the 52 VHH antibodies tested for neutralizing activity against SVA, 7 VHH antibodies did not exhibit neutralizing activity against SVA. The 7 VHH antibodies were VHH-3, VHH-9, VHH-11, VHH-24, VHH-31, VHH-49, and VHH-51. The remaining 45 VHH antibodies exhibited neutralizing activity. Among the 45 VHH antibodies, the following exhibited high neutralizing activity: VHH-2, VHH-4, VHH-5, VHH-8, VHH-10, VHH-15, VHH-27, VHH-30, VHH-34, VHH-42, VHH-47, and VHH-52. Furthermore, antibodies VHH-2, VHH-5, VHH-10, VHH-27, and VHH-47 displayed the highest neutralizing activity.

TABLE 5
Neutralizing activity of VHH antibodies

| Antibody ID | SVA |
|---|---|
| 1 | 1:32 |
| 2 | 1:512 |
| 3 | 0 |
| 4 | 1:128 |
| 5 | 1:512 |
| 6 | 1:64 |
| 7 | 1:16 |
| 8 | 1:128 |
| 9 | 0 |
| 10 | 1:512 |
| 11 | 0 |
| 12 | 1:8 |
| 13 | 1:64 |
| 14 | 1:64 |
| 15 | 1:128 |
| 16 | 1:32 |
| 17 | 1:64 |
| 18 | 1:32 |
| 19 | 1:32 |
| 20 | 1:4 |
| 21 | 1:16 |
| 22 | 1:16 |
| 23 | 1:4 |
| 24 | 0 |
| 25 | 1:64 |
| 26 | 1:64 |
| 27 | 1:512 |
| 28 | 1:32 |
| 29 | 1:32 |
| 30 | 1:128 |
| 31 | 0 |
| 32 | 1:8 |
| 33 | 1:16 |
| 34 | 1:128 |
| 35 | 1:32 |
| 36 | 1:64 |
| 37 | 1:4 |
| 38 | 1:4 |
| 39 | 1:8 |
| 40 | 1:4 |
| 41 | 1:4 |
| 42 | 1:128 |
| 43 | 1:64 |
| 44 | 1:8 |
| 45 | 1:4 |
| 46 | 1:16 |
| 47 | 1:512 |
| 48 | 1:4 |
| 49 | 0 |
| 50 | 1:32 |
| 51 | 0 |
| 52 | 1:128 |
| Fc | 0 |
| PBS | 0 |

It will be obvious to those skilled in the art that changes and modifications may be made, and therefore, the aim in the appended claims is to cover all such changes and modifications.

What is claimed is:

1. A single-domain antibody or an antigen-binding fragment thereof to Senecavirus A, the single-domain antibody or the antigen-binding fragment thereof being selected from 52 amino acid sequence groups, and each of the 52 amino acid sequence groups comprising a first variable domain of heavy chain VH-CDR-1, a second variable domain of heavy chain VH-CDR-2, and a third variable domain of heavy chain VH-CDR-3; the first variable domain of heavy chain VH-CDR-1 having an amino acid sequence represented by one of SEQ ID NO: 1-SEQ ID NO: 52, the second variable domain of heavy chain VH-CDR-2 having an amino acid sequence represented by one of SEQ ID NO: 53-SEQ ID NO: 104, and the third variable domain of heavy chain VH-CDR-3 having an amino acid sequence represented by one of SEQ ID NO: 105-SEQ ID NO: 156.

2. The single-domain antibody or the antigen-binding fragment thereof of claim 1, wherein the first variable domain of heavy chain VH-CDR-1 has an amino acid sequence represented by SEQ ID NO: 2, SEQ ID NO: 4, SEQ ID NO: 5, SEQ ID NO: 8, SEQ ID NO: 10, SEQ ID NO: 15, SEQ ID NO: 27, SEQ ID NO: 30, SEQ ID NO: 34, SEQ ID NO: 42, SEQ ID NO: 47, or SEQ ID NO: 52; the second variable domain of heavy chain VH-CDR-2 has an amino acid sequence represented by SEQ ID NO: 54, SEQ ID NO: 56, SEQ ID NO: 57, SEQ ID NO: 60, SEQ ID NO: 62, SEQ ID NO: 67, SEQ ID NO: 79, SEQ ID NO: 82, SEQ ID NO: 86, SEQ ID NO: 94, SEQ ID NO: 99, or SEQ ID NO: 104; and the third variable domain of heavy chain VH-CDR-3 has an amino acid sequence represented by SEQ ID NO: 106, SEQ ID NO: 108, SEQ ID NO: 109, SEQ ID NO: 112, SEQ ID NO: 114, SEQ ID NO: 119, SEQ ID NO: 131, SEQ ID NO: 134, SEQ ID NO: 138, SEQ ID NO: 146, SEQ ID NO: 151, or SEQ ID NO: 156.

3. The single-domain antibody or the antigen-binding fragment thereof of claim 1, wherein the first variable domain of heavy chain VH-CDR-1 has an amino acid sequence represented by SEQ ID NO: 2, SEQ ID NO: 5, SEQ ID NO: 10, SEQ ID NO: 27, or SEQ ID NO: 47; the second variable domain of heavy chain VH-CDR-2 has an amino acid sequence represented by SEQ ID NO: 54, SEQ ID NO: 57, SEQ ID NO: 62, SEQ ID NO: 79, or SEQ ID NO: 99; and the third variable domain of heavy chain VH-CDR-3 has an amino acid sequence represented by SEQ ID NO: 106, SEQ ID NO: 109, SEQ ID NO: 114, SEQ ID NO: 131, or SEQ ID NO: 151.

4. The single-domain antibody or the antigen-binding fragment thereof of claim 1, wherein the single-domain antibody is isolated from camelid single-domain antibody.

5. A nucleic acid molecule encoding the single-domain antibody or antigen-binding fragment thereof of claim 1.

6. A vector or host cell comprising the nucleic acid molecule of claim 5.

7. A chimeric antibody comprising the single-domain antibody or antigen-binding fragment thereof of claim 1.

8. A composition, comprising the single-domain antibody or antigen-binding fragment thereof of claim 1, a nucleic acid molecule encoding the single-domain antibody, a vector or host cell comprising the nucleic acid molecule, and/or a chimeric antibody comprising the single-domain antibody.

9. A method for diagnosis, prevention, and/or treatment of viral infections comprising administering a patient in need thereof the single-domain antibody or antigen-binding fragment thereof of claim 1, a nucleic acid molecule encoding the single-domain antibody, a vector or host cell comprising the nucleic acid molecule, a chimeric antibody comprising the single-domain antibody, and/or a composition comprising the single-domain antibody.

10. A kit for detecting Seneca virus A, comprising an instruction manual and a reagent for detecting the single-domain antibody or antigen-binding fragment thereof of claim 1, a nucleic acid molecule encoding the single-domain antibody, a vector or host cell comprising the nucleic acid molecule, a chimeric antibody comprising the single-domain antibody, and/or a composition comprising the single-domain antibody.


SEQUENCE LISTING (160 sequences)

SEQ ID NO: 1 - protein, 9 aa, synthetic construct
YIKDINRLG

SEQ ID NO: 2 - protein, 9 aa, synthetic construct
YTDSRFCLA

SEQ ID NO: 3 - protein, 9 aa, synthetic construct
VTTCSNNMS

SEQ ID NO: 4 - protein, 16 aa, synthetic construct
YAYIRYIGYSSSYCMG

SEQ ID NO: 5 - protein, 10 aa, synthetic construct
YTFSSRPCMG

SEQ ID NO: 6 - protein, 10 aa, synthetic construct
NTYSNTEYMG

SEQ ID NO: 7 - protein, 9 aa, synthetic construct
YTYSSNCMG

SEQ ID NO: 8 - protein, 9 aa, synthetic construct
VIYSSACLA

SEQ ID NO: 9 - protein, 10 aa, synthetic construct
STDCSTYDMR

SEQ ID NO: 10 - protein, 9 aa, synthetic construct
YTYCNYDMN

SEQ ID NO: 11 - protein, 4 aa, synthetic construct
YCIG

SEQ ID NO: 12 - protein, 9 aa, synthetic construct
PTYSTDCIG

SEQ ID NO: 13 - protein, 9 aa, synthetic construct
YTYRSYCMG

SEQ ID NO: 14 - protein, 9 aa, synthetic construct
YPYSSNCMG

SEQ ID NO: 15 - protein, 9 aa, synthetic construct
LIFSDCTMA

SEQ ID NO: 16 - protein, 9 aa, synthetic construct
YTYSSNCMG

SEQ ID NO: 17 - protein, 9 aa, synthetic construct
VATSRLSMA

SEQ ID NO: 18 - protein, 9 aa, synthetic construct
YTYSSNCMG

SEQ ID NO: 19 - protein, 9 aa, synthetic construct
YIASLNCMA

SEQ ID NO: 20 - protein, 9 aa, synthetic construct
YTFSSNCVG

SEQ ID NO: 21 - protein, 9 aa, synthetic construct
FTARSYCMG

SEQ ID NO: 22 - protein, 9 aa, synthetic construct
YQFSNGHVA

SEQ ID NO: 23 - protein, 9 aa, synthetic construct
YTYSSGCMG

SEQ ID NO: 24 - protein, 9 aa, synthetic construct
HTYCGNDRS

SEQ ID NO: 25 - protein, 9 aa, synthetic construct
YTYKRRCMG

SEQ ID NO: 26 - protein, 9 aa, synthetic construct
YPSSNHCMG

SEQ ID NO: 27 - protein, 9 aa, synthetic construct
YIERHYCMG

SEQ ID NO: 28 - protein, 9 aa, synthetic construct
YTVSSDCMG

SEQ ID NO: 29 - protein, 9 aa, synthetic construct
FTSSINGMA

SEQ ID NO: 30 - protein, 9 aa, synthetic construct
DTYSTVCLG

SEQ ID NO: 31 - protein, 11 aa, synthetic construct
FRFIYKDYCMG

SEQ ID NO: 32 - protein, 9 aa, synthetic construct
YTDSSYCMG

SEQ ID NO: 33 - protein, 9 aa, synthetic construct
FTFSTYHMG

SEQ ID NO: 34 - protein, 9 aa, synthetic construct
ATYATDCMG

SEQ ID NO: 35 - protein, 9 aa, synthetic construct
YMSGNYCVG

SEQ ID NO: 36 - protein, 9 aa, synthetic construct
FSYNNKCVA

SEQ ID NO: 37 - protein, 9 aa, synthetic construct
YISSVSCMG

SEQ ID NO: 38 - protein, 9 aa, synthetic construct
YAASANCMA

SEQ ID NO: 39 - protein, 7 aa, synthetic construct
YDFSCMG

SEQ ID NO: 40 - protein, 9 aa, synthetic construct
SIFSSCRMG

SEQ ID NO: 41 - protein, 9 aa, synthetic construct
FPYSTVCMG

SEQ ID NO: 42 - protein, 9 aa, synthetic construct
YIYSRSCMG

SEQ ID NO: 43 - protein, 9 aa, synthetic construct
YMDNTYCIG

SEQ ID NO: 44 - protein, 9 aa, synthetic construct
YTYSTHCMG

SEQ ID NO: 45 - protein, 9 aa, synthetic construct
YSFSGYCIG

SEQ ID NO: 46 - protein, 9 aa, synthetic construct
YIYSASCMG

SEQ ID NO: 47 - protein, 9 aa, synthetic construct
YISNDYCMG

SEQ ID NO: 48 - protein, 9 aa, synthetic construct
YPSSNHCMG

SEQ ID NO: 49 - protein, 9 aa, synthetic construct
YTLSPYCMG

SEQ ID NO: 50 - protein, 9 aa, synthetic construct
YTYCNYDMS

SEQ ID NO: 51 - protein, 9 aa, synthetic construct
FTFSHYDMR

SEQ ID NO: 52 - protein, 9 aa, synthetic construct
DTYSTVCLG

SEQ ID NO: 53 - protein, 18 aa, synthetic construct
TIRTVVGGSTYYTDSVKG

SEQ ID NO: 54 - protein, 16 aa, synthetic construct
SIDSSGMTTYADSVKG

SEQ ID NO: 55 - protein, 15 aa, synthetic construct
SIGVGTARYADSVKG

SEQ ID NO: 56 - protein, 16 aa, synthetic construct
TIEPNGSASYSDSVKG

SEQ ID NO: 57 - protein, 16 aa, synthetic construct
LIGRDGSTWYANSVKG

SEQ ID NO: 58 - protein, 18 aa, synthetic construct
TIRMVVGGSTYYADSVKG

SEQ ID NO: 59 - protein, 17 aa, synthetic construct
AIYTAGGNTYYADSVKG

SEQ ID NO: 60 - protein, 16 aa, synthetic construct
RIDGYGNTRYEDSVKG

SEQ ID NO: 61 - protein, 16 aa, synthetic construct
QSRTDGTTTYANAVKG

SEQ ID NO: 62 - protein, 16 aa, synthetic construct
GLVYGGSTTYADSVKG

SEQ ID NO: 63 - protein, 17 aa, synthetic construct
SIHVATGITYSADSVKG

SEQ ID NO: 64 - protein, 16 aa, synthetic construct
QIASDGARRYADSVKG

SEQ ID NO: 65 - protein, 17 aa, synthetic construct
TVFIGGDSTYYADSVKG

SEQ ID NO: 66 - protein, 16 aa, synthetic construct
SIDSGGGTTYSASALE

SEQ ID NO: 67 - protein, 17 aa, synthetic construct
SINADGGTPTYTDSVKG

SEQ ID NO: 68 - protein, 17 aa, synthetic construct
AIYTGTGSTYYVDSVKG

SEQ ID NO: 69 - protein, 17 aa, synthetic construct
AVNAGDGGTYYADSVKG

SEQ ID NO: 70 - protein, 17 aa, synthetic construct
TIHTEYDSPYYASAVKG

SEQ ID NO: 71 - protein, 17 aa, synthetic construct
YIGSGDGSTDYADSVKG

SEQ ID NO: 72 - protein, 16 aa, synthetic construct
RYDGSGDPRYADSVKG

SEQ ID NO: 73 - protein, 16 aa, synthetic construct
GIGFDGSTNYADSAKG

SEQ ID NO: 74 - protein, 15 aa, synthetic construct
AIYAGGPVYTDSVKG

SEQ ID NO: 75 - protein, 16 aa, synthetic construct
TSDSDGRTSYADSVKG

SEQ ID NO: 76 - protein, 16 aa, synthetic construct
SIDSVGNTWYADSVKG

SEQ ID NO: 77 - protein, 17 aa, synthetic construct
SIYTGGSSTYYADSVKG

SEQ ID NO: 78 - protein, 16 aa, synthetic construct
SIDSVGITTYADSVLG

SEQ ID NO: 79 - protein, 16 aa, synthetic construct
TVAYEGSTTYAESVKG

SEQ ID NO: 80 - protein, 16 aa, synthetic construct
SIDNIGQTNYGDSVKG

SEQ ID NO: 81 - protein, 17 aa, synthetic construct
AISSDGGVTDYADSVKG

SEQ ID NO: 82 - protein, 16 aa, synthetic construct
VIDVDGKIVYADSIEG

SEQ ID NO: 83 - protein, 16 aa, synthetic construct
VIDSDGRTAYANYVAG

SEQ ID NO: 84 - protein, 16 aa, synthetic construct
AIGSDGGTSYADSVKG

SEQ ID NO: 85 - protein, 16 aa, synthetic construct
AIDKYGTTYYRDSVKG

SEQ ID NO: 86 - protein, 15 aa, synthetic construct
AYSRGTALYDDSVRG

SEQ ID NO: 87 - protein, 16 aa, synthetic construct
VIEGGGSTRYAAAVKD

SEQ ID NO: 88 - protein, 17 aa, synthetic construct
FIYTGDSSTDYFDSVKG

SEQ ID NO: 89 - protein, 17 aa, synthetic construct
VISAADGSTDYDDSVKG

SEQ ID NO: 90 - protein, 16 aa, synthetic construct
SIYVGGRTYYGDSVKG

SEQ ID NO: 91 - protein, 16 aa, synthetic construct
SIRSFGGVYYADTVKG

SEQ ID NO: 92 - protein, 16 aa, synthetic construct
SVSDDGITTYAEAVKG

SEQ ID NO: 93 - protein, 17 aa, synthetic construct
TIYTAFGSTYYADSVKG

SEQ ID NO: 94 - protein, 17 aa, synthetic construct
TIYTGSGSPWYADSVKG

SEQ ID NO: 95 - protein, 16 aa, synthetic construct
AIGSVGSTSYADSVKG

SEQ ID NO: 96 - protein, 17 aa, synthetic construct
YYYTGSGSTYYTDSVKG

SEQ ID NO: 97 - protein, 16 aa, synthetic construct
RFDRDGRTTYAESVKG

SEQ ID NO: 98 - protein, 17 aa, synthetic construct
VISPGDGSTDYADSVKG

SEQ ID NO: 99 - protein, 17 aa, synthetic construct
ALHTGFGSTYYPDSVKG

SEQ ID NO: 100 - protein, 16 aa, synthetic construct
SIDSVGTTTYADSVLG

SEQ ID NO: 101 - protein, 16 aa, synthetic construct
VIDSTGSTKYAHSVKG

SEQ ID NO: 102 - protein, 16 aa, synthetic construct
GIESFGDTSYADSVKG

SEQ ID NO: 103 - protein, 17 aa, synthetic construct
GINSDGGDTYYADSVKG

SEQ ID NO: 104 - protein, 16 aa, synthetic construct
VIDVDGKIVYADSIEG

SEQ ID NO: 105 - protein, 15 aa, synthetic construct
GLGLYPSLSAAEYNY

SEQ ID NO: 106 - protein, 17 aa, synthetic construct
RSRAWYEECSWVNKYSW

SEQ ID NO: 107 - protein, 11 aa, synthetic construct
FCPNGGLWSGY

SEQ ID NO: 108 - protein, 16 aa, synthetic construct
RAAINDCYGAPGNFDY

SEQ ID NO: 109 - protein, 16 aa, synthetic construct
DPEYSASWCRDRRYRA

SEQ ID NO: 110 - protein, 15 aa, synthetic construct
AMGLYPSLSRNEYNY

SEQ ID NO: 111 - protein, 20 aa, synthetic construct
EIDPIWSTFTCGDWEADFSY

SEQ ID NO: 112 - protein, 16 aa, synthetic construct
STTGWSCPVSLRMFEY

SEQ ID NO: 113 - protein, 15 aa, synthetic construct
VGFGCRTGTVRNFNY

SEQ ID NO: 114 - protein, 13 aa, synthetic construct
TTRGGWCPSGYDY

SEQ ID NO: 115 - protein, 17 aa, synthetic construct
PTTYYCPSWEYESMFRW

SEQ ID NO: 116 - protein, 19 aa, synthetic construct
RRGHPVWGCRAPDFDDYNF

SEQ ID NO: 117 - protein, 17 aa, synthetic construct
QTGYTGSWCTEVASYGY

SEQ ID NO: 118 - protein, 18 aa, synthetic construct
GERACFWNCFRTGWRNAD

SEQ ID NO: 119 - protein, 11 aa, synthetic construct
YYCRFLGFSSF

SEQ ID NO: 120 - protein, 20 aa, synthetic construct
RVPRSSSSWCSSLRRDEYVY

SEQ ID NO: 121 - protein, 15 aa, synthetic construct
YFLGGDNLSRSQYNY

SEQ ID NO: 122 - protein, 16 aa, synthetic construct
KAWGGDWCSRLDNYNY

SEQ ID NO: 123 - protein, 15 aa, synthetic construct
DRISSTWCRFREYVS

SEQ ID NO: 124 - protein, 14 aa, synthetic construct
GWALGWPATCDYSY

SEQ ID NO: 125 - protein, 18 aa, synthetic construct
DFFRGSWCTGERAPTLRN

SEQ ID NO: 126 - protein, 16 aa, synthetic construct
GLFFGAIWYDELRYFY

SEQ ID NO: 127 - protein, 16 aa, synthetic construct
GLFFGAIWYDELRYFY

SEQ ID NO: 128 - protein, 12 aa, synthetic construct
VCTGGSGPTKPV

SEQ ID NO: 129 - protein, 14 aa, synthetic construct
KSDYWTWCNDGYSD

SEQ ID NO: 130 - protein, 18 aa, synthetic construct
GSRAWYENCGRTVRRYSD

SEQ ID NO: 131 - protein, 15 aa, synthetic construct
RTTYFCTPRANDFTY

SEQ ID NO: 132 - protein, 19 aa, synthetic construct
QQFCTVGSRLSLDRDWYNY

SEQ ID NO: 133 - protein, 17 aa, synthetic construct
DWYGSWYEVRRTKAYYY

SEQ ID NO: 134 - protein, 18 aa, synthetic construct
GRKRHWALPTCGGMRPDY

SEQ ID NO: 135 - protein, 19 aa, synthetic construct
TPVHPVFGQCRLVAGAFDY

SEQ ID NO: 136 - protein, 21 aa, synthetic construct
NPGYLYSVVGYCYPAAPRNTH

SEQ ID NO: 137 - protein, 16 aa, synthetic construct
SPGYGGPWYDSTWYNL

SEQ ID NO: 138 - protein, 14 aa, synthetic construct
GNRAWYERSCQYND

SEQ ID NO: 139 - protein, 18 aa, synthetic construct
RSRAWYESSCGNVGRFTW

SEQ ID NO: 140 - protein, 16 aa, synthetic construct
SLAPSGSWCRARYYGY

SEQ ID NO: 141 - protein, 18 aa, synthetic construct
DRRCYSGSWCLWRREYNY

SEQ ID NO: 142 - protein, 18 aa, synthetic construct
SVSGGCWNFHPPFYEWRY

SEQ ID NO: 143 - protein, 15 aa, synthetic construct
DNSYYCDVDKYDHKY

SEQ ID NO: 144 - protein, 14 aa, synthetic construct
DHYHLIFRHNLCGR

SEQ ID NO: 145 - protein, 16 aa, synthetic construct
ALLGTCCTDLLDGYNY

SEQ ID NO: 146 - protein, 16 aa, synthetic construct
EPSFCTRSRRESDFRY

SEQ ID NO: 147 - protein, 17 aa, synthetic construct
RFRSWYESCYTNQKYIY

SEQ ID NO: 148 - protein, 22 aa, synthetic construct
LRAGYGGSWYECAISRANEYVH

SEQ ID NO: 149 - protein, 21 aa, synthetic construct
VRPLCYRYCPPLGGAASDYNQ

SEQ ID NO: 150 - protein, 18 aa, synthetic construct
DRRGSSGSWCLWRRAYSW

SEQ ID NO: 151 - protein, 18 aa, synthetic construct
ARYSGSWCIVRGAYNHGY

SEQ ID NO: 152 - protein, 18 aa, synthetic construct
GPRAWYENCGRTVRRYNA

SEQ ID NO: 153 - protein, 26 aa, synthetic construct
APGRPTDLNTCMIAIEAPGAMRSAEI

SEQ ID NO: 154 - protein, 13 aa, synthetic construct
NTRGGWCPSGFDY

SEQ ID NO: 155 - protein, 8 aa, synthetic construct
GTIGISEP

SEQ ID NO: 156 - protein, 18 aa, synthetic construct
GRKRHWALPTCGGMRPDY

SEQ ID NO: 157 - DNA, 23 bp, synthetic construct
  source 1..23: mol_type = other DNA
ggtacgtgctgttgaactgttcc

SEQ ID NO: 158 - DNA, 65 bp, synthetic construct
  source 1..65: mol_type = other DNA
aacatgccatgactcgcggctcaaccggccatggctgakgtbcagctgcaggcgtctggrggagg

SEQ ID NO: 159 - DNA, 53 bp, synthetic construct
  source 1..53: mol_type = other DNA
gttattattattcagattattagtgcggccgctggagacggtgaccwgggtcc

SEQ ID NO: 160 - DNA, 23 bp, synthetic construct
  source 1..23: mol_type = other DNA
gtcctggctgctcttctacaagg